(19) United States
(12) Patent Application Publication
(10) Pub. No.: US 2006/0019989 A1
(43) Pub. Date: Jan. 26, 2006

(54) Compositions comprising 5-alpha reductase inhibitors and SERMs and methods of use thereof

(76) Inventors: Mitchell S. Steiner, Germantown, TN (US); Karen A. Veverka, Cordova, TN (US); Duane D. Miller, Germantown, TN (US)

Correspondence Address: EITAN, PEARL, LATZER & COHEN ZEDEK LLP, 10 ROCKEFELLER PLAZA, SUITE 1001, NEW YORK, NY, 10020, US

(21) Appl. No.: 10/895,401

(22) Filed: Jul. 21, 2004

(51) Int. Cl. A61K 31/138; A61K 31/47

(52) U.S. Cl. 514/310; 514/651

(57) ABSTRACT

This invention provides for combinations of 5 alpha reductase inhibitors and SERMs. These combinations are useful in: 1) preventing prostate carcinogenesis in a subject; 2) preventing the recurrence of, suppressing, inhibiting or reducing the incidence of prostate carcinogenesis in a subject; 3) treating a subject with prostate cancer; 4) suppressing, inhibiting or reducing the incidence of prostate cancer in a subject; 5) treating a subject with pre-malignant lesions of prostate cancer; 6) suppressing, inhibiting or reducing the incidence of pre-malignant lesions of prostate cancer in a subject; 7) reducing the incidence, inhibiting, suppressing, preventing and/or treating androgen-deprivation induced conditions in men suffering from prostate cancer, such as androgen-deprivation induced osteoporosis, bone fractures, loss of bone mineral density (BMD), hot flashes and/or gynecomastia; and 8) treating polycystic ovarian syndrome and reducing the incidence, inhibiting, suppressing, preventing and/or treating diabetes, cardiovascular disease, breast cancer and endometrial cancer in women suffering from polycystic ovarian syndrome.

### CROSS REFERENCE TO RELATED APPLICATIONS

This Application claims priority of U.S. Ser. No. 10/305,363, filed Nov. 27, 2002, U.S. Ser. No. 10/611,056, filed Nov. 8, 2000, and U.S. Ser. No. 10/300,939, filed Nov. 21, 2002, which are hereby incorporated by reference.

### FIELD OF INVENTION

This invention relates to combinations of a 5 alpha reductase inhibitor and a selective estrogen receptor modulator. The combinations are useful for 1) preventing prostate carcinogenesis in a subject; 2) preventing the recurrence of, suppressing, inhibiting or reducing the incidence of prostate carcinogenesis in a subject; 3) treating a subject with prostate cancer; 4) suppressing, inhibiting or reducing the incidence of prostate cancer in a subject; 5) treating a subject with pre-malignant lesions of prostate cancer; 6) suppressing, inhibiting or reducing the incidence ofpre-malignant lesions of prostate cancer in a subject; 7) reducing the incidence, inhibiting, suppressing, preventing and/or treating androgen-deprivation induced conditions in men suffering from prostate cancer, such as androgen-deprivation induced osteoporosis, bone fractures, loss of bone mineral density (BMD), hot flashes and/or gynecomastia; and 8) treating polycystic ovarian syndrome and reducing the incidence, inhibiting, suppressing, preventing and/or treating diabetes, cardiovascular disease, breast cancer and endometrial cancer in women suffering from polycystic ovarian syndrome.

### BACKGROUND OF THE INVENTION

Prostate cancer is one of the most frequently occurring cancers among men in the United States, with hundreds of thousands of new cases diagnosed each year. Unfortunately, over sixty percent of newly diagnosed cases of prostate cancer are found to be pathologically advanced, with no cure and a dismal prognosis. One approach to this problem is to find prostate cancer earlier through screening programs and thereby reduce the number of advanced prostate cancer patients. Another strategy, however, is to develop drugs to prevent prostate cancer. One third of all men over 50 years of age have a latent form of prostate cancer that may be activated into the life-threatening clinical prostate cancer form. The frequency of latent prostatic tumors has been shown to increase substantially with each decade of life from the 50 s (5.3-14%) to the 90 s (40-80%). The number of people with latent prostate cancer is the same across all cultures, ethnic groups, and races, yet the frequency of clinically aggressive cancer is markedly different. This suggests that environmental factors may play a role in activating latent prostate cancer. Thus, the development of chemoprevention strategies against prostate cancer may have the greatest overall impact both medically and economically against prostate cancer.

Because of the high incidence and mortality of prostate cancer, it is imperative that effective which factors contribute to prostate carcinogenesis, including the initiation, promotion, and progression of prostate cancer, will provide molecular mechanistic clues as to appropriate points of intervention to prevent or halt the carcinogenic process. New innovative approaches are urgently needed at both the basic science and clinical levels to decrease the incidence of prostate cancer as well as to halt or cause the regression of latent prostate cancer. As the frequency of prostate cancer escalates dramatically at the same ages at which men are confronted by other competing causes of mortality, simply slowing the progression of prostate adenocarcinoma may be both a more suitable and a cost effective health strategy.

Various approaches have been taken to the chemoprevention of prostate cancer. Greenwald, “Expanding Horizons in Breast and Prostate Cancer Prevention and Early Detection” in J. Cancer Education, 1993, Vol. 8, No. 2, pages 91-1 07, discusses the testing of 5a-reductase inhibitors such as finasteride for the prevention of prostate cancer. Brawley et al., “Chemoprevention of Prostate Cancer” in Urology, 1994, Vol. 43, No. 5, also mentions 5a-reductase inhibitors as well as difluoromethylornithine and retinoids as potential chemopreventive agents.

Kelloff et al., “Introductory Remarks: Development of Chemopreventive Agents for Prostate Cancer” in Journal of Cellular Biochemistry, 1992, Supplement 16H: 1-8, describes National Cancer Institute preclinical studies of seven agents: alltrans-N-(4-hydroxyphenyl) retinamide, difluoromethylornithine, dehydroepiandrosterone, liarozole, lovastatin, oltipraz, and finasteride.

Lucia et al., “Chemopreventive Activity of Tamoxifen, N-(4-Hydroxyphenyl) retinamide, and the Vitamin D Analogue Ro24-553 1 for Androgen-promoted Carcinomas of the Rat Seminal Vesicle and Prostate” in Cancer Research, 1995, Vol. 55, pages 5621-5627, reports chemoprevention of prostate carcinomas in Lobund-Wistar rats by tamoxifen, an estrogen response modifier.

As discussed in Pofter et al., “A mechanistic hypothesis for DNA adduct formation by tamoxifen following hepatic oxidative metabolism” in Carcinogenesis, 1994, Vol. 15, No. 3, pages 439-442, tamoxifen causes liver carcinogenicity in rats, which is attributed to the formation of covalent DNA adducts. This reference also reports that the tamoxifen analogue toremifene, which showed a much lower level of hepatic DNA adduct formation than tamoxifen, is non-carcinogenic.

Toremifene is an example of a triphenylalkene compound described in U.S. Pat. Nos. 4,696,949 and 5,491,173 to Toivola et al., the disclosures of which are incorporated herein by reference. The parenteral and topical administration to mammalian subjects of formulations containing toremifene are described in U.S. Pat. No. 5,571,534 to Jalonen et al. and in U.S. Pat. No. 5,605,700 to DeGregorio et al., the disclosures of which are incorporated herein by reference.

Toremifene-containing formulations for reversing the multidrug resistance to cancer cells to a cytotoxic drug are described in U.S. Pat. No. 4,990,538 to Harris et al., the disclosure of which is incorporated herein by reference.

U.S. Pat. Nos. 5,595,722 and 5,599,844 to Grainger et al., the disclosures of which are incorporated herein by reference, describe methods for identifying agents that increase TGFP levels and for orally administering formulations containing TGFP activators and TGFP production stimulators to prevent or treat conditions characterized by abnormal proliferation of smooth muscle cells, for example, vascular trauma. Disclosed agents for increasing TGFP levels include tamoxifen and its analogue toremifene.

U.S. Pat. Nos. 5,629,007 and 5,635,197 to Audia et al., the disclosures of which are incorporated herein by reference, describe a method of preventing the development of prostatic cancer in a patient at risk of developing such cancer, for example, a patient having benign prostatic hyperplasia, by administering to the patient an octahydrobenzo [f} quinolin-3-one compound.

U.S. Pat. No. 5,595,985 to Labrie, the disclosure of which is incorporated herein by reference, also describes a method for treating benign prostatic hyperplasia using a combination of a 5a-reductase inhibitor and a compound that binds and blocks access to androgen receptors. One example of a compound that blocks androgen receptors is flutamide.

U.S. Pat. Nos. 4,329,364 and 4,474,813 to Neri et al., the disclosures of which are incorporated herein by reference, describe pharmaceutical compositions comprising flutamide for delaying and/or preventing the onset of prostate carcinoma. The preparation can be in the form of a capsule, tablet, suppository, or elixir.

Despite these developments, there is a continuing need for agents and methods effective for preventing prostate cancer. The present invention is directed to satisfying this need.

In addition to a need for an optimal treatment for prostate cancer, of the approaches to the treatment of prostate cancer, the most commonly utilized, androgen deprivation therapy, is fraught with significant side effects, including hot flashes, gynecomastia, osteoporosis, decreased lean muscle mass, depression and other mood changes, loss of libido, and erectile dysfunction [Stege R (2000), Prostate Suppl 10,38-42]. Consequently, complications of androgen blockade now contribute significantly to the morbidity, and in some cases the mortality, of men suffering from prostate cancer.

Given that more patients today are being treated by long-term androgen deprivation, osteoporosis in particular has become a clinically important side effect of men suffering from prostate cancer undergoing androgen deprivation. Loss of bone mineral density (BMD) occurs in the majority of patients being treated by androgen deprivation by 6 months. New innovative approaches are urgently needed at both the basic science and clinical levels to decrease the incidence of androgen-deprivation induced osteoporosis in men suffering from prostate cancer.

It is also well established that the bone mineral density of males in general decreases with age. Decreased amounts of bone mineral content and density correlates with decreased bone strength and predisposes to fracture. While our understanding of the molecular mechanisms underlying the pleiotropic effects of sex-hormones in non-reproductive tissues is not yet complete, nonetheless, physiologic concentrations of androgens and estrogens appear to play an important role in maintaining bone homeostasis throughout the life-cycle. Consequently, when androgen or estrogen deprivation occurs, there is a resultant increase in the rate of bone remodeling that tilts the balance of resorption and formation in the favor of resorption, contributing to an overall loss of bone mass. In males, the natural decline in sex-hormones at maturity (direct decline in androgens as well as lower levels of estrogens derived from peripheral aromatization of androgens) is associated with the frailty of bones. This effect is also observed in males who have been castrated.

Polycystic Ovarian Syndrome (PCOS) is characterized by menstrual irregularity and hirsutism and is a common cause of anovulatory infertility. The biochemical abnormalities are a high concentration of plasma luteinising hormone (LH) or a high LH/follicle stimulating hormone (FSH) ratio and high concentrations of estrogen and androgens (testosterone and/or androstenedione and/or dehydroepiandrosterone (DHEA), which are secreted by the ovary and/or the adrenal gland. Clinical manifestations of PCOS include amenorrhea, hirsutism acanthosis nigricans, acne and obesity. Women with PCOS are typically hirsute, infertile, and present with an increased risk, and/or early onset of diabetes and cardiovascular disease.

Thus, there remains a need for the development of therapies that address the clinical conditions discussed above, and others which are a result of the modulation of sex-steroid levels as a result of aging, disease or medical intervention.

### SUMMARY OF THE INVENTION

In one embodiment, this invention provides for combinations of a 5-alpha reductase inhibitor and a selective estrogen receptor modulator (SERM).

In one embodiment, this invention provides a composition comprising a 5-alpha reductase inhibitor and a selective estrogen receptor modulator (SERM) compound represented by the structure of formula I, its N-oxide, ester, pharmaceutically acceptable salt, hydrate, or any combination thereof: 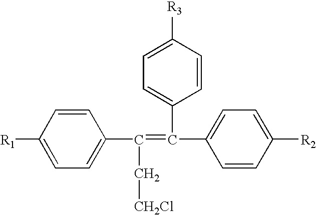
wherein R1 and R2, which can be the same or different, are H or OH; R3 is OCH2CH2NR4R5, wherein R4 and R5, which can be the same or different, are H or an alkyl group of 1 to about 4 carbon atoms.

In one embodiment, the selective estrogen receptor modulator compound is an analog, isomer, metabolite, derivative, pharmaceutically acceptable salt, pharmaceutical product, N-oxide, hydrate or any combination thereof of said compound of formula I.

In one embodiment, the selective androgen receptor modulator compound is triphenylethylene, toremifene, or a combination thereof.

In one embodiment, the composition comprises a compound of formula I, or an analog or a metabolite thereof at a concentration of 5 mg, or in another embodiment, 50 mg, or in another embodiment, 500 mg.

In one embodiment, the 5-alpha reductase inhibitor is dutasteride or finasteride, or a combination thereof.

In one embodiment, this invention provides a method of suppressing, inhibiting, or reducing the incidence of pre-malignant lesions of prostate cancer in a subject comprising the step of administering to said subject a composition of this invention, in an amount effective to suppress, inhibit or reduce the incidence of pre-malignant lesions of prostate cancer in the subject.

In another embodiment, this invention provides a method of treating a human with pre-malignant lesions of prostate cancer in a subject comprising the step of administering to said subject a composition of this invention, in an amount effective to treat of pre-malignant lesions of prostate cancer in the subject.

In another embodiment, this invention provides a method of suppressing, inhibiting, or reducing the incidence of latent prostate cancer in a subject comprising the step of administering to said subject a composition of this invention, in an amount effective to suppress, inhibit or reduce the incidence of prostate cancer in the subject.

In another embodiment, this invention provides a method of treating a subject with latent prostate cancer comprising the step of administering to said subject a composition of this invention, in an amount effective to treat prostate cancer in the subject.

According to this aspect of the invention, suppressing, inhibiting, reducing the incidence of or treating prostate cancer is via suppressing, inhibiting, reducing the incidence of or treating a precancerous precursor of prostate adenocarcinoma, wherein, in one embodiment, precancerous precursor of prostate adenocarcinoma is prostate intraepithelial neoplasia (PIN), and in another embodiment, the prostate intraepithelial neoplasia is high grade prostate intraepithelial neoplasia (HGPIN).

In another embodiment, this invention provides a method of preventing suppressing, inhibiting, or reducing the incidence of prostate carcinogenesis in a subject comprising the step of administering to said subject a composition of this invention, in an amount effective to prevent, suppress, inhibit or reduce the incidence of prostate cancer in the subject.

In another embodiment, this invention provides a method of treating androgen-deprivation induced osteoporosis in a male subject suffering from prostate cancer, comprising the step of administering to said subject a composition of this invention, in an amount effective to treat androgen-deprivation induced osteoporosis in said subject.

In another embodiment, this invention provides a method of suppressing, inhibiting, reducing the risk of developing or preventing androgen-deprivation induced osteoporosis in a male subject suffering from prostate cancer, comprising the step of administering to said subject the composition of claim 1, in an amount effective to suppress, inhibit, reduce the risk of developing or prevent androgen-deprivation induced osteoporosis in said subject.

In another embodiment, this invention provides a method of suppressing, inhibiting, reducing the risk of developing, preventing or treating androgen-deprivation induced loss of bone mineral density (BMD) in a male subject suffering from prostate cancer, comprising the step of administering to said subject a composition of this invention, in an amount effective to suppress, inhibit, reduce the risk of developing, prevent or treat androgen-deprivation induced bone loss in said subject.

In another embodiment, this invention provides a method of suppressing, inhibiting, reducing the risk of developing, preventing or treating androgen-deprivation induced bone fractures in a male subject suffering from prostate cancer, comprising the step of administering to said subject a composition of this invention, in an amount effective to suppress, inhibit, reduce the risk of developing, prevent or treat androgen-deprivation induced bone fractures in said subject.

In another embodiment, this invention provides a method of suppressing, inhibiting, reducing the risk of developing, preventing or treating a subject with hot flashes, comprising the step of administering to said subject a composition of this invention, in an amount effective to suppress, inhibit, reduce the risk of developing, prevent or treat hot flashes in said subject.

According to this aspect of the invention, and in one embodiment, the subject suffers from prostate cancer and has been exposed to androgen-deprivation therapy.

In another embodiment, this invention provides a method of suppressing, inhibiting, reducing the risk of developing, preventing or treating a subject with gynecomastia, comprising the step of administering to said subject a composition of this invention, in an amount effective to suppress, inhibit, reduce the risk of developing, prevent or treat gynecomastia in said subject.

According to this aspect of the invention, and in one embodiment, the subject suffers from prostate cancer and has been exposed to androgen-deprivation therapy.

In another embodiment, this invention provides a method of suppressing, inhibiting, reducing the risk of developing, preventing or treating a subject with endometrial carcinoma, comprising the step of administering to said subject a composition of this invention, in an amount effective to suppress, inhibit, reduce the risk of developing, prevent or treat endometrial carcinoma in said subject.

In another embodiment, this invention provides a method of suppressing, inhibiting, reducing the risk of developing, preventing or treating a subject with polycystic ovarian syndrome, comprising the step of administering to said subject a composition of this invention, in an amount effective to suppress, inhibit, reduce the risk of developing, prevent or treat polycystic ovarian syndrome in said subject.

In another embodiment, this invention provides a method of suppressing, inhibiting, delaying onset or preventing diabetes, breast cancer, endometrial carcinoma or cardiovascular disease in a female subject suffereing from polycystic ovarian syndrome, comprising the step of administering to said subject a composition of this invention, in an amount effective to suppress, inhibit, delay onset, or prevent diabetes, breast cancer, endometrial carcinoma or cardiovascular disease in the subject.

### DETAILED DESCRIPTION OF THE INVENTION

This invention provides for combinations of 5 alpha reductase inhibitors and SARMs. Such combinations are useful in: 1) preventing prostate carcinogenesis in a subject; 2) preventing the recurrence of, suppressing, inhibiting or reducing the incidence of prostate carcinogenesis in a subject; 3) treating a subject with prostate cancer; 4) suppressing, inhibiting or reducing the incidence of prostate cancer in a subject; 5) treating a subject with pre-malignant lesions of prostate cancer; 6) suppressing, inhibiting or reducing the incidence of pre-malignant lesions of prostate cancer in a subject; 7) reducing the incidence, inhibiting, suppressing, preventing and/or treating androgen-deprivation induced conditions in men suffering from prostate cancer, such as androgen-deprivation induced osteoporosis, bone fractures, loss of bone mineral density (BMD), hot flashes and/or gynecomastia; and 8) treating polycystic ovarian syndrome and reducing the incidence, inhibiting, suppressing, preventing and/or treating diabetes, cardiovascular disease, breast cancer and endometrial cancer in women suffering from polycystic ovarian syndrome.

In one embodiment, this invention provides a composition comprising a 5-alpha reductase inhibitor and a selective estrogen receptor modulator (SERM) compound represented by the structure of formula I, its N-oxide, ester, pharmaceutically acceptable salt, hydrate, or any combination thereof: 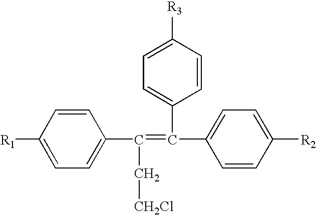
wherein R1 and R2, which can be the same or different, are H or OH; R3 is OCH2CH2NR4R5, wherein R4 and R5, which can be the same or different, are H or an alkyl group of 1 to about 4 carbon atoms.

In one embodiment, the compound of formula I will have R1 and R2 groups, which are the same, or in another embodiment, are different. In one embodiment, R1 and R2 may be H, or in another embodiment, OH. In another embodiment, R3 is OCH₂CH₂N. The substituents R4 or R5 are defined herein as being the same or, in another embodiment, different, which in one embodiment is an H or, in another embodiment, an alkyl group of 1 to about 4 carbon atoms.

An “alkyl” group refers to a saturated aliphatic hydrocarbon, including straight-chain, branched-chain and cyclic alkyl groups. In one embodiment, the alkyl group has 1-12 carbons. In another embodiment, the alkyl group has 1-7 carbons. In another embodiment, the alkyl group has 1-6 carbons. In another embodiment, the alkyl group has 1-4 carbons. The alkyl group may be unsubstituted or substituted by one or more groups selected from halogen, hydroxy, alkoxy carbonyl, amido, alkylamido, dialkylamido, nitro, amino, alkylamino, dialkylamino, carboxyl, thio and thioalkyl.

In one embodiment, this invention provides a composition comprising a 5-alpha reductase inhibitor and a SERM compound, which is an analog of the compound of formula I, or in another embodiment, a derivative of the compound of formula I, or in another embodiment, an isomer of the compound of formula I, or in another embodiment, a metabolite of the compound of formula I, or in another embodiment, a pharmaceutically acceptable salt of the compound of formula I, or in another embodiment, a pharmaceutical product of the compound of formula I, or in another embodiment, a hydrate of the compound of formula I, or in another embodiment, an N-oxide of the compound of formula I, or in another embodiment, a combination of any of an analog, derivative, metabolite, isomer, pharmaceutically acceptable salt, pharmaceutical product, hydrate or N-oxide of the compound of formula I.

In one embodiment, the term “isomer” includes, but is not limited to, optical isomers and analogs, structural isomers and analogs, conformational isomers and analogs, and the like.

In one embodiment, this invention encompasses the use of various optical isomers of the SERM compound. It will be appreciated by those skilled in the art that the SERMs of the present invention contain at least one chiral center.

Accordingly, the SERMs used in the compositions and methods of the present invention may exist in, and be isolated in, optically-active or racemic forms. Some compounds may also exhibit polymorphism. It is to be understood that the present invention encompasses any racemic, optically-active, polymorphic, or stereroisomeric form, or mixtures thereof, which form possesses properties useful in the treatment of androgen-related conditions described herein. In one embodiment, the SERMs are the pure (R)-isomers. In another embodiment, the SERMs are the pure (S)-isomers. In another embodiment, the SERMs are a mixture of the (R) and the (S) isomers. In another embodiment, the SERMs are a racemic mixture comprising an equal amount of the (R) and the (S) isomers. It is well known in the art how to prepare optically-active forms (for example, by resolution of the racemic form by recrystallization techniques, by synthesis from optically-active starting materials, by chiral synthesis, or by chromatographic separation using a chiral stationary phase).

The invention includes pharmaceutically acceptable salts of amino-substituted compounds with organic and inorganic acids, for example, citric acid and hydrochloric acid. The invention also includes N-oxides of the amino substituents of the compounds described herein. Pharmaceutically acceptable salts can also be prepared from the phenolic compounds by treatment with inorganic bases, for example, sodium hydroxide. Also, esters of the phenolic compounds can be made with aliphatic and aromatic carboxylic acids, for example, acetic acid and benzoic acid esters.

This invention further includes derivatives of the SERM compounds. The term “derivatives” includes but is not limited to ether derivatives, acid derivatives, amide derivatives, ester derivatives and the like. In addition, this invention further includes hydrates of the SERM compounds. The term “hydrate” includes but is not limited to hemihydrate, monohydrate, dihydrate, trihydrate and the like.

This invention further includes metabolites of the SERM compounds. The term “metabolite” means any substance produced from another substance by metabolism or a metabolic process.

This invention further includes pharmaceutical products of the SERM compounds. The term “pharmaceutical product” means a composition suitable for pharmaceutical use (pharmaceutical composition), as defined herein.

In one embodiment, the compositions may comprise the following SERMs in combination with a 5 alpha reductase inhibitor (5-ARI): triphenylalkylenes such as triphenylethylenes, which include Tamoxifen, Droloxifene, Toremifene, Idoxifene, Clomiphene, Enclomiphene and Zuclomiphene; benzothiphene derivatives such as Raloxifene and LY 353381; benzopyran derivatives such as EM 800 (SCH 57050) and its metabolite EM 652; naphthalene derivatives such as Lasofoxifene (CP 336,156); chromans such as Levormeloxifene or their analogs, raloxifene, derivatives, isomers, or metabolites thereof, or their pharmaceutically acceptable salts, esters, N-oxides, or mixtures thereof.

Toremifene is an example of a triphenylalkylene compound described in U.S. Pat. Nos. 4,696,949 and 5,491,173 to Toivola et al., the disclosures of which are incorporated herein by reference. The parenteral and topical administration to mammalian subjects of formulations containing Toremifene is described in U.S. Pat. No. 5,571,534 to Jalonen et al. and in U.S. Pat. No. 5,605,700 to DeGregorio et al., the disclosures of which are incorporated herein by reference.

On administration, toremifene has several metabolites that are also biologically active, which are well known to those skilled in the art, which are also useful for the applications listed herein, including, and representing embodiments thereof, treating, preventing, preventing recurrence of, suppressing, and/or inhibiting prostate cancer and for treating, preventing, preventing recurrence of, suppressing, and/or inhibiting pre-malignant lesions of prostate cancer. These analogs and/or metabolites include but are not limited to 4-chloro-1,2-diphenyl-1-[4-[2-(N-methylamino) ethoxy]phenyl]-1-butene; 4-chloro-1,2-diphenyl-1-[4-[2-(N,N-diethylamino) ethoxy]phenyl]-1-butene; 4-chloro-1,2-diphenyl-1-[4 (aminoethoxy)]-1-butene; 4-chloro-1-(4-hydroxyphenyl)-1-[4-[2-(N,N-dimethylamino) ethoxy] phenyl]-2-phenyl-1-butene; 4-chloro-1-(4-hydroxyphenyl)-1-[4-[2-(N-methylamino)ethoxy] phenyl]-2-phenyl-1-butene; and 4-chloro-1,2-bis(4-hydroxyphenyl)-1-[4-[2-(N,N-dimethylamino)ethoxy]phenyl]-1-butene. It is to be understood that any SERM metabolite when formulated in a composition in combination with a 5-ARI is to be considered as part of this invention, as is its use for the applications described herein.

The compositions of this invention will have effective amounts of the 5-ARI and SERM together with suitable diluents, preservatives, solubilizers, emulsifiers, adjuvants, and/or carriers. An “effective amount” refers, in one embodiment, to that amount which provides a desired effect for a given application, as described further hereinunder, and in another embodiment, may be a function of administration regimen. Such compositions are liquids or lyophilized or otherwise dried formulations and include diluents of various buffer content (e.g., Tris-HCl., acetate, phosphate), pH and ionic strength, additives such as albumin or gelatin to prevent absorption to surfaces, detergents (e.g., Tween 20, Tween 80, Pluronic F68, bile acid salts), solubilizing agents (e.g., glycerol, polyethylene glycerol), anti-oxidants (e.g., ascorbic acid, sodium metabisulfite), preservatives (e.g., Thimerosal, benzyl alcohol, parabens), bulking substances or tonicity modifiers (e.g., lactose, mannitol), covalent attachment of polymers such as polyethylene glycol to the protein, complexation with metal ions, or incorporation of the material into or onto particulate preparations of polymeric compounds such as polylactic acid, polglycolic acid, hydrogels, etc., or onto liposomes, microemulsions, micelles, unilamellar or multilamellar vesicles, erythrocyte ghosts, or spheroplasts. Such compositions will influence the physical state, solubility, stability, rate of in vivo release, and rate of in vivo clearance. Controlled or sustained release compositions include formulation in lipophilic depots (e.g., fatty acids, waxes, oils). Also comprehended by the invention are particulate compositions coated with polymers (e.g., poloxamers or poloxamines). Other embodiments of the compositions of the invention incorporate particulate forms, protective coatings, protease inhibitors, or permeation enhancers for various routes of administration, including parenteral, pulmonary, nasal, and oral. In one embodiment, the pharmaceutical composition is administered parenterally, paracancerally, transmucosally, transdermally, intramuscularly, intravenously, intradermally, subcutaneously, intraperitonealy, intraventricularly, intracranially, or intratumorally.

The dosage of each compound may be in the range of 0.1-80 mg/day. In one embodiment the dosage is in the range of 5-50, or in another embodiment, 5-100, or in another embodiment, 5-500 mg/day. In another embodiment, the dosage is in the range of 35-66 mg/day. In another embodiment the dosage is in the range of 40-60 mg/day. In another embodiment the dosage is in a range of 45-60 mg/day. In another embodiment the dosage is in the range of 15-25 mg/day. In another embodiment the dosage is in the range of 55-65 mg/day. In another embodiment the dosage is in the range of 45-60 mg/day. In another embodiment the dosage is in the range of 60-80 mg/day. In another embodiment the dosage is 20 mg/day. In another embodiment the dosage is 40 mg/day. In another embodiment the dosage is 60 mg/day. In another embodiment the dosage is 80 mg/day.

In one embodiment, the SERM, or an analog or a metabolite thereof is at a dosage of 20 mg, or in another embodiment, 40 mg, or in another embodiment, 60 mg.

The compositions of this invention comprise a 5 alpha reductase inhbitor in combination with a SERM. In one embodiment, the 5 alpha reductase inhibitor is MK-906, a product of Merck, Sharp & Dohme (Mc Connell et al., J. Urol. 141: 239A, 1989). In another embodiment, the 5 alpha reductase inhibitor is 17β-N,N-diethylcarbamoyl-4-methyl-4-aza-5.alpha.-androstan-3-one (4-MA) (Brooks et al., Endocrinology 109: 830, 1981; Liang et al., Endocrinology 112: 1460, 1983). In another embodiment, the 5 alpha reductase inhibitor is a 4-azasteroid, which can be formed as in Liang et al., J. Biol. chem. 259: 734-739, 1984; and in Brooks et al., Steroids 47: 1-19, 1986.). In another embodiment, the 5 alpha reductase inhibitor is a 6-methylene-4-pregnene-3,20-dione, for example, as described (Petrow et al., J. Endocrinol. 95: 311-313, 1982). In another embodiment, the 5 alpha reductase inhibitor is a 4-methyl-3-oxo-4-aza-5.alpha.-pregnane-30(s) carboxylate (Kadohama et al., J. Natl. Cancer Inst. 74: 475-486, 1985).

The enzyme 5.alpha.-reductase catalyzes the conversion of testosterone to dihydrotestosterone (DHT), and an inhibitor of this enzyme prevents the conversion such that it selectively reduces DHT levels without reducing testosterone levels.

One of the principal mediators of androgenic activity in a target organ is 5.alpha.-dihydrotestosterone, which in many cases is a far more potent androgen than testosterone itself, and is formed locally in the target organ by the action of testosterone-5.alpha.-reductase. Inhibitors of testosterone-5.alpha.-reductase prevent or lessen symptoms of hyperandrogenic stimulation, and its combination with SERMs will, in one embodiment, serve to treat diseases, disorders and conditions which are stimulated, exacerbated or prolonged by elevated androgen production, accompanied in one embodiment by elevated estrogen production.

In another embodiment, the composition will comprise a pharmaceutically acceptable carrier. Such carriers are well known to those skilled in the art and include, but are not limited to, 0.01-0.1M and preferably 0.05M phosphate buffer or 0.8% saline. Additionally, such pharmaceutically acceptable carriers may be aqueous or non-aqueous solutions, suspensions, or emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions, and suspensions, including saline and buffered media. Parenteral vehicles include sodium chloride solution, Ringer's dextrose, dextrose and sodium chloride, lactated Ringer's, and fixed oils. Intravenous vehicles include fluid and nutrient replenishers, electrolyte replenishers such as those based on Ringer's dextrose, and the like. Preservatives and other additives may also be present, such as, for example, antimicrobials, antioxidants, collating agents, inert gases, and the like.

Controlled or sustained release compositions include formulation in lipophilic depots (e.g. fatty acids, waxes, oils). Also comprehended by the invention are particulate compositions coated with polymers (e.g. poloxamers or poloxamines) and the compound coupled to antibodies directed against tissue-specific receptors, ligands, or antigens or coupled to ligands of tissue-specific receptors. Other embodiments of the compositions of the invention incorporate particulate forms, protective coatings, protease inhibitors, or permeation enhancers for various routes of administration, including parenteral, pulmonary, nasal, and oral. Compounds modified by the covalent attachment of water-soluble polymers such as polyethylene glycol, copolymers of polyethylene glycol and polypropylene glycol, carboxymethyl cellulose, dextran, polyvinyl alcohol, polyvinylpyrrolidone, or polyproline, are known to exhibit substantially longer half-lives in blood following intravenous injection than do the corresponding unmodified compounds (Abuchowski et al., 1981; Newmark et al., 1982; and Katre et al., 1987). Such modifications may also increase the compound's solubility in aqueous solution, eliminate aggregation, enhance the physical and chemical stability of the compound, and greatly reduce the immunogenicity and reactivity of the compound. As a result, the desired in vivo biological activity may be achieved by the administration of such polymer-compound abducts less frequently or in lower doses than with the unmodified compound.

In yet another embodiment, the composition can be delivered in a controlled release system. For example, the SERM and 5-ARI may be administered using intravenous infusion, an implantable osmotic pump, a transdermal patch, liposomes, or other modes of administration. In one embodiment, a pump may be used (see Langer, supra; Sefton, CRC Crit. Ref. Biomed. Eng. 14:201 (1987); Buchwald et al., Surgery 88:507 (1980); Saudek et al., N. Engl. J. Med. 321:574 (1989). In another embodiment, polymeric materials can be used. In yet another embodiment, a controlled release system can be placed in proximity to the therapeutic target, i.e., the brain, thus requiring only a fraction of the systemic dose (see, e.g., Goodson, in Medical Applications of Controlled Release, supra, vol. 2, pp. 115-138 (1984). Preferably, a controlled release device is introduced into a subject in proximity of the site of inappropriate immune activation or a tumor. Other controlled release systems are discussed in the review by Langer (Science 249:1527-1533 (1990).

The compositions of this invention can be in solid or liquid form such as tablets, powders, capsules, pellets, solutions, suspensions, elixirs, emulsions, gels, creams, or suppositories, including rectal and urethral suppositories. Pharmaceutically acceptable carriers used may include gums, starches, sugars, cellulosic materials, and mixtures thereof. The compositions of this invention may be administered to a subject by, for example, subcutaneous implantation of a pellet; in a further embodiment, the pellet provides for controlled release of active agent over a period of time. The preparation can also be administered by intravenous, intraarterial, or intramuscular injection of a liquid preparation, oral administration of a liquid or solid preparation, or by topical application. Administration can also be accomplished by use of a rectal suppository or a urethral suppository. The composition can also be a parenteral formulation; in one embodiment, the formulation comprises a liposome that includes a complex of a active agents such as, for example, toremifene, 5-ARI and a cyclodextrin compound, as described (U.S. Pat. No. 5,571,534 to Jalonen et al).

The compositions of the invention can be prepared by known dissolving, mixing, granulating, or tablet-forming processes. For oral administration, the compounds of the present invention or their physiologically tolerated derivatives such as salts, esters, N-oxides, and the like are mixed with additives customary for this purpose, such as vehicles, stabilizers, or inert diluents, and converted by customary methods into a suitable form for administration, such as tablets, coated tablets, hard or soft gelatin capsules, aqueous, alcoholic, or oily solutions. Examples of suitable inert vehicles are conventional tablet bases such as lactose, sucrose, or cornstarch in combination with binders like acacia, cornstarch, gelatin, or with disintegrating agents such as cornstarch, potato starch, alginic acid, or with a lubricant such as stearic acid or magnesium stearate. Examples of suitable oily vehicles or solvents are vegetable or animal oils such as sunflower oil or fish-liver oil. Preparations can be effected as dry or as wet granules. For parenteral administration (subcutaneous, intravenous, intraarterial, or intramuscular injection), the compounds of the present invention or their physiologically tolerated derivatives such as salts, esters, N-oxides, and the like are converted into a solution, suspension, or emulsion, if desired, with the substances customary and suitable for this purpose, for example, solubilizers or other auxiliaries. Examples are: sterile liquids such as water and oils, with or without the addition of a surfactant and other pharmaceutically acceptable adjuvants. Illustrative oils are those of petroleum, animal, vegetable, or synthetic origin, for example, peanut oil, soybean oil, or mineral oil. In general, water, saline, aqueous dextrose, and related sugar solutions, and glycols such as propylene glycols or polyethylene glycol are preferred liquid carriers, particularly for injectable solutions.

The preparation of compositions that contain the active components is well understood in the art. Such compositions may be prepared as an aerosol for delivery to the nasopharynx or as injectables, either as liquid solutions or suspensions, although solid forms suitable for solution in, or suspension in, liquid prior to injection can also be prepared. The preparation can also be emulsified. The active ingredients may be mixed with excipients that are pharmaceutically acceptable and compatible with the active ingredients. Suitable excipients are, for example, water, saline, dextrose, glycerol, ethanol, or the like and combinations thereof. In addition, if desired, the composition can contain minor amounts of auxiliary substances such as wetting or emulsifying agents, or pH buffering agents, which enhance the effectiveness of the active ingredient.

Active components can be formulated into the composition as neutralized pharmaceutically acceptable salt forms. Pharmaceutically acceptable salts include the acid addition salts (formed with the free amino groups of the polypeptide or antibody molecule) and are formed with inorganic acids such as, for example, hydrochloric or phosphoric acids, or such organic acids as acetic, oxalic, tartaric, mandelic, and the like. Salts formed from the free carboxyl groups can also be derived from inorganic bases such as, for example, sodium, potassium, ammonium, calcium, or ferric hydroxides, and such organic bases as isopropylamine, trimethylamine, 2-ethylamino ethanol, histidine, procaine, and the like.

For topical administration to body surfaces using, for example, creams, gels, drops, and the like, the active agents or their physiologically tolerated derivatives such as salts, esters, N-oxides, and the like are prepared and applied as solutions, suspensions, or emulsions in a physiologically acceptable diluent with or without a pharmaceutical carrier.

In another embodiment, the active compounds can be delivered in a vesicle, in particular a liposome (see Langer, Science 249:1527-1533 (1990); Treat et al., in Liposomes in the Therapy of Infectious Disease and Cancer, Lopez-Berestein and Fidler (eds.), Liss, New York, pp. 353-365 (1989); Lopez-Berestein, ibid., pp. 317-327; see generally ibid).

In one embodiment, this invention provides a method of suppressing, inhibiting, or reducing the incidence of pre-malignant lesions of prostate cancer in a subject comprising the step of administering to said subject a composition of this invention, in an amount effective to suppress, inhibit or reduce the incidence of pre-malignant lesions of prostate cancer in the subject.

In another embodiment, this invention provides a method of treating a human with pre-malignant lesions of prostate cancer in a subject comprising the step of administering to said subject a composition of this invention, in an amount effective to treat of pre-malignant lesions of prostate cancer in the subject.

In another embodiment, this invention provides a method of suppressing, inhibiting, or reducing the incidence of latent prostate cancer in a subject comprising the step of administering to said subject a composition of this invention, in an amount effective to suppress, inhibit or reduce the incidence of prostate cancer in the subject.

In another embodiment, this invention provides a method of treating a subject with latent prostate cancer comprising the step of administering to said subject a composition of this invention, in an amount effective to treat prostate cancer in the subject.

In another embodiment, this invention provides a method of preventing the recurrence of, suppressing, inhibiting or reducing the incidence of prostate carcinogenesis, or increasing the survival rate of a subject having prostate cancer, or preventing prostate carcinogenesis, comprising the step of administering to said subject a composition of this invention, in an amount effective to suppress, inhibit or reduce the incidence of prostate carcinogenesis, increase the survival rate of a subject having prostate cancer or prevent prostate carcinogenesis.

According to this aspect of the invention, suppressing, inhibiting, reducing the incidence of or treating prostate cancer is via suppressing, inhibiting, reducing the incidence of or treating a precancerous precursor of prostate adenocarcinoma, wherein, in one embodiment, precancerous precursor of prostate adenocarcinoma is prostate intraepithelial neoplasia (PIN), and in another embodiment, the prostate intraepithelial neoplasia is high grade prostate intraepithelial neoplasia (HGPIN).

In one embodiment, the subject has an elevated risk of prostate cancer. In another embodiment, the subject has benign prostatic hyperplasia, prostatic intraepithelial neoplasia (PIN), or an abnormally high level of circulating prostate specific antibody (PSA).

In another embodiment, this invention provides a method of preventing suppressing, inhibiting, or reducing the incidence of prostate carcinogenesis in a subject comprising the step of administering to said subject a composition of this invention, in an amount effective to prevent, suppress, inhibit or reduce the incidence of prostate cancer in the subject.

In one embodiment, the prostate cancer is latent prostate cancer. In another embodiment, the subject has a precancerous precursors of prostate adenocarcinoma. In another embodiment, the precancerous precursors of prostate adenocarcinoma is prostate intraepithelial neoplasia (PIN). In another embodiment, the prostate intraepithelial neoplasia is high grade prostate intraepithelial neoplasia (HGPIN).

A variety of chemical compounds, also described as “chemotherapeutic agents”, function to induce DNA damage, in rapidly dividing cells, thus utilized as a treatment regimen for neoplastic cells. In one embodiment, the compositions of this invention may be administered in parallel with such chemotherapeutic agents, for example, adriamycin, 5-fluorouracil (5FU), etoposide (VP-16), camptothecin, actinomycin-D, mitomycin C, cisplatin (CDDP) and even hydrogen peroxide. The invention also encompasses the use of a combination of one or more DNA-damaging agents, whether radiation-based or actual compounds, such as the use of X-rays with cisplatin or the use of cisplatin with etoposide.

In another embodiment, one may irradiate the localized tumor site with DNA-damaging radiation such as X-rays, UV-light, gamma-rays, or even microwaves. Alternatively, the tumor cells may be contacted with the DNA-damaging agent by administering to the subject a therapeutically effective amount of a pharmaceutical composition comprising a DNA-damaging compound, such as adriamycin, 5-fluorouracil, etoposide, camptothecin, actinomycin-D, mitomycin C, or more preferably, cisplatin. Agents that damage DNA also include compounds that interfere with DNA replication, mitosis, and chromosomal segregation. Such chemotherapeutic compounds include adriamycin, also known as doxorubicin, etoposide, verapamil, podophyllotoxin, and the like.

Intermediate endpoint biomarkers are measurable biologic alterations in tissue that occur between the initiation of and the development of frank neoplasia. A biomarker is validated if the final endpoint, cancer incidence, is also reduced by the putative compounds of the present invention. Intermediate biomarkers in cancer may be classified into the following groups: histologic, proliferation, differentiation, and biochemical markers. In any chemoprevention strategy, the availability of histologically recognizable and accepted precancerous lesions constitutes an important starting point. For the prostate, a histological marker is a precancerous precursor of prostatic adenocarcinoma, of which prostatic intraepithelial neoplasia (PIN) is an example. PIN appears as an abnormal proliferation within the prostatic ducts of premalignant foci of cellular dysplasia and carcinoma in situ without stromal invasion. PIN and histological prostate cancer are morphometrically and phenotypically similar. Thus, the development of high-grade PIN represents an important step in the progression pathway whereby the normal prostate develops PIN, histological prostate cancer, invasive clinical prostate cancer, and metastases. It is to be understood that treatment regimens of this invention contemplate efficacy determinations via frank changes in biomarker expression. In another embodiment, changes in biomarker expression represent preventive therapy.

Prostate intraepithelial neoplasia has been shown to be a precancerous lesion, or precursor of prostatic adenocarcinoma. Prostate intraepithelial neoplasia is the abnormal proliferation within the prostatic ducts of premalignant foci of cellular dysplasia and carcinoma in situ without stromal invasion. Prostate intraepithelial neoplasia is the most accurate and reliable marker of prostate carcinogenesis and may be used as an acceptable endpoint in prostate chemoprevention trials. Prostate intraepithelial neoplasia has a high predictive value as a marker for adenocarcinoma, and its identification warrants repeat biopsy for concurrent or subsequent invasive carcinoma. Most studies suggest that most patients with prostate intraepithelial neoplasia will develop carcinoma within 10 years. Interestingly, prostate intraepithelial neoplasia does not contribute to serum PSA, which is not surprising, since, unlike prostate cancer, prostate intraepithelial neoplasia has not yet invaded the vasculature of the prostate to leak PSA into the blood stream. Thus, prostate intraepithelial neoplasia may precede even prostate-cancer related serum PSA elevations. It is to be understood that any effect upon prostate carcinogenesis by the compositions of this invention are to be considered as part of the invention.

In one embodiment, the compositions of the present invention comprise at least one 5 alpha reductase inhibitor (5-ARI) and at least one SERM compound as the active ingredients, however it is to be understood that multiple 5-ARI and SERM compounds may be utilized in the methods of this invention, and compositions comprising the same are to be considered as part of this invention. In another embodiment of this invention, the compositions and methods of use thereof may further comprise one or more therapeutic agents. These agents include, but are not limited to: LHRH/GnRH agonists, reversible antiandrogens, antiestrogens, anticancer drugs, aromatase inhibitors, progestins, agents acting through other nuclear hormone receptors, selective androgen receptor modulators (SARMs), progesterone, estrogen, PDE5 inhibitors, apomorphine, bisphosphonate, sulfonurea compounds, statins or combinations thereof.

Osteoporosis is a systemic skeletal disease, characterized by low bone mass and deterioration of bone tissue, with a consequent increase in bone fragility and susceptibility to fracture. In osteoporotic patients, bone strength is abnormal, with a resulting increase in the risk of fracture. Osteoporosis depletes both the calcium and the protein collagen normally found in the bone, resulting in either abnormal bone quality or decreased bone density. Bones that are affected by osteoporosis can fracture with only a minor fall or injury that normally would not cause a bone fracture. The fracture can be either in the form of cracking (as in a hip fracture) or collapsing (as in a compression fracture of the spine). The spine, hips, and wrists are common areas of osteoporosis bone fractures, although fractures can also occur in other skeletal areas.

BMD is a measured calculation of the true mass of bone. The absolute amount of bone as measured by bone mineral density (BMD) generally correlates with bone strength and its ability to bear weight. By measuring BMD, it is possible to predict fracture risk in the same manner that measuring blood pressure can help predict the risk of stroke.

BMD in one embodiment can be measured by known bone-mineral content mapping techniques. Bone density of the hip, spine, wrist, or calcaneus may be measured by a variety of techniques. The preferred method of BMD measurement is dual-energy x-ray densitometry (DXA). BMD of the hip, antero-posterior (AP) spine, lateral spine, and wrist can be measured using this technology. Measurement at any site predicts overall risk of fracture, but information from a specific site is the best predictor of fracture at that site. Quantitative computerized tomography (QCT) is also used to measure BMD of the spine. See for example, “Nuclear Medicine: “Quantitative Procedures”. by Wahner H W, Dunn W L, Thorsen H C, et al, published by Toronto Little, Brown & Co., 1983, (see pages 107-132). An article entitled “Assessment of Bone Mineral Part 1” appeared in the Journal of Nuclear Medicine, pp 1134-1141, (1984). Another article entitled “Bone Mineral Density of The Radius” appeared in Vol. 26, No. 11, (1985) Nov. Journal of Nuclear Medicine at pp 13-39. Abstracts on the use of gamma cameras for bone-mineral content measurements are (a) S. Hoory et al, Radiology, Vol. 157(P), p. 87 (1985), and (b) C. R. Wilson et al, Radiology, Vol. 157(P), p. 88 (1985).

The present invention provides a safe and effective method for treating, preventing, suppressing, inhibiting or reducing the risk of developing androgen-deprivation induced osteoporosis and/or loss of BMD and is particularly useful for treating male subjects suffering from prostate cancer having an elevated risk of developing androgen-deprivation induced osteoporosis. In one embodiment, the male subject is a mammalian subject. In another embodiment, the male subject is a human subject.

Furthermore, the compositions presented herein are effective at treating, suppressing or inhibiting osteopenia accompanied by bone loss. “Osteopenia” refers to decreased calcification or density of bone. This is a term, which encompasses all skeletal systems in which such a condition is noted.

Accordingly, the present invention provides a method of treating androgen-deprivation induced osteoporosis in a male subject suffering from prostate cancer, the method comprising the step of administering to said subject a composition of this invention, in an amount effective to treat androgen-deprivation induced osteoporosis in said subject.

In another embodiment, this invention provides a method of suppressing, inhibiting, reducing the risk of developing or preventing androgen-deprivation induced osteoporosis in a male subject suffering from prostate cancer, comprising the step of administering to said subject the composition of claim 1, in an amount effective to suppress, inhibit, reduce the risk of developing or prevent androgen-deprivation induced osteoporosis in said subject.

In another embodiment, this invention provides a method of suppressing, inhibiting, reducing the risk of developing, preventing or treating androgen-deprivation induced loss of bone mineral density (BMD) in a male subject suffering from prostate cancer, comprising the step of administering to said subject a composition of this invention, in an amount effective to suppress, inhibit, reduce the risk of developing, prevent or treat androgen-deprivation induced bone loss in said subject.

In another embodiment, this invention provides a method of suppressing, inhibiting, reducing the risk of developing, preventing or treating androgen-deprivation induced bone fractures in a male subject suffering from prostate cancer, comprising the step of administering to said subject a composition of this invention, in an amount effective to suppress, inhibit, reduce the risk of developing, prevent or treat androgen-deprivation induced bone fractures in said subject.

The term “treating”, in one embodiment, includes preventative as well as disorder remitative treatment. The terms “reducing”, “suppressing” and “inhibiting” have their commonly understood meaning of lessening or decreasing, in another embodiment. The term “progression” means, in another embodiment, increasing in scope or severity, advancing, growing or becoming worse. The term “recurrence” means, in another embodiment, the return of a disease after a remission.

The term “administering”, in another embodiment, refers to bringing a subject in contact with an anti-estrogen compound of the present invention. Administration can be accomplished in vitro, i.e. in a test tube, or in vivo, i.e. in cells or tissues of living organisms, for example humans. In one embodiment, the present invention encompasses administering the compounds of the present invention to a subject.

In another embodiment, this invention provides a method of suppressing, inhibiting, reducing the risk of developing, preventing or treating a subject with hot flashes, comprising the step of administering to said subject a composition of this invention, in an amount effective to suppress, inhibit, reduce the risk of developing, prevent or treat hot flashes in said subject.

According to this aspect of the invention, and in one embodiment, the subject suffers from prostate cancer and has been exposed to androgen-deprivation therapy.

In another embodiment, this invention provides a method of suppressing, inhibiting, reducing the risk of developing, preventing or treating a subject with gynecomastia, comprising the step of administering to said subject a composition of this invention, in an amount effective to suppress, inhibit, reduce the risk of developing, prevent or treat gynecomastia in said subject.

According to this aspect of the invention, and in one embodiment, the subject suffers from prostate cancer and has been exposed to androgen-deprivation therapy.

In another embodiment, this invention provides a method of suppressing, inhibiting, reducing the risk of developing, preventing or treating a subject with endometrial carcinoma, comprising the step of administering to said subject a composition of this invention, in an amount effective to suppress, inhibit, reduce the risk of developing, prevent or treat endometrial carcinoma in said subject.

In another embodiment, this invention provides a method of suppressing, inhibiting, reducing the risk of developing, preventing or treating a subject with polycystic ovarian syndrome, comprising the step of administering to said subject a composition of this invention, in an amount effective to suppress, inhibit, reduce the risk of developing, prevent or treat polycystic ovarian syndrome in said subject.

In another embodiment, this invention provides a method of suppressing, inhibiting, delaying onset or preventing diabetes, breast cancer, endometrial carcinoma or cardiovascular disease in a female subject suffereing from polycystic ovarian syndrome, comprising the step of administering to said subject a composition of this invention, in an amount effective to suppress, inhibit, delay onset, or prevent diabetes, breast cancer, endometrial carcinoma or cardiovascular disease in the subject.

The following examples are presented in order to more fully illustrate the preferred embodiments of the invention. They should in no way, however, be construed as limiting the broad scope of the invention.

### EXAMPLE 1

### 5-ARI and SERM Compositions

A Tablet formulation, with scored tablets for oral use, may be prepared containing, in one embodiment, 500 mg. of each active ingredient. The tablets may be prepared, in one embodiment, from the following ingredients:

| | Gm. |
|---|---|
| 17β-N,N--diethylcarbamoyl-4-methyl-4-aza-5.alpha.- | 5000 |
| androstan-3-one | |
| Toremifene | 5000 |
| Starch, U.S.P. | 350 |
| Talc, U.S.P. | 250 |
| Calcium stearate | 35 |

The active ingredients are granulated with a 4% w./v. aqueous solution of methylcellulose U.S.P. (1500 cps). Tablets containing 0.1, 1, 5, 10, 15, 25, 50, and 100 mg. of each active ingredient may also be prepared, in other embodiments, by substituting 1, 10, 50, 100, 150, 250, 500, and 1000 gm. of 2500 gm. in the above formulation. To the dried granules is added a mixture of the remainder of the ingredients and the final mixture compressed into tablets of proper weight.

Capsules—hard gelatin capsules for oral use, each containing 250 mg. of active ingredients may be prepared, in another embodiment from the following ingredients:

| | Gm |
|---|---|
| 17β-N,N--diethylcarbamoyl-4-methyl-4-aza-5.alpha.- | 2500 |
| androstan-3-one | |
| Toremifene | 2500 |
| Lactose, U.S.P. | 1000 |
| Starch, U.S.P. | 300 |
| Talc, U.S.P. | 65 |
| Calcium Stearate | 25 |

The active ingredients are mixed with the starch lactose mixture followed by the talc and calcium stearate. The final mixture is then encapsulated in the usual manner. Capsules containing 0.1, 1, 5, 10, 15, 25, 50, and 100 mg. of each active ingredient is also prepared by substituting 1, 10, 50, 100, 150, 250, 500, and 1000 gm. of 2500 gm. in the above formulation. In another embodiment, the concentration of the SERM is 10, or in another embodiment 25, or in another embodiment 50% that of the 5-ARI, in any composition of this invention.

Soft elastic capsules—One-piece soft elastic capsules for oral use, each containing 500 mg. of each, or 250 mg of each active material are prepared in the usual manner by first dispersing the active material in sufficient corn oil to render the material capsulatable.

Aqueous suspension—An aqueous suspension for oral use containing in each 5 ml., 0.25 g. of each active ingredient is prepared from the following ingredients:

| | Gm. |
|---|---|
| 17β-N,N--diethylcarbamoyl-4-methyl-4-aza-5.alpha.- | 500 |
| androstan-3-one | |
| Toremifene | 500 |
| Methylparaben, U.S.P. | 7.5 |
| Propylparaben, U.S.P. | 2.5 |
| Saccharin sodium | 12.5 |
| Glycerin | 3000 |
| Tragacanth powder | 10 |
| Orange oil flavor | 10 |
| F.D. & C. orange dye | 7.5 |
| Deionized water, q.s. to 10,000 ml | |

It will be appreciated by a person skilled in the art that the present invention is not limited by what has been particularly shown and described hereinabove. Rather, the scope of the invention is defined by the claims that follow:

What is claimed is:

1. A composition comprising a 5-alpha reductase inhibitor and a selective estrogen receptor modulator (SERM) compound represented by the structure of formula I, its N-oxide, ester, pharmaceutically acceptable salt, hydrate, or any combination thereof: 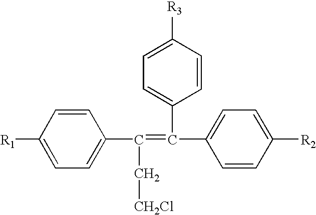
wherein R1 and R2, which can be the same or different, are H or OH; R3 is OCH2CH2NR4R5, wherein R4 and R5, which can be the same or different, are H or an alkyl group of 1 to about 4 carbon atoms.

2. The composition according to claim 1, wherein said selective estrogen receptor modulator compound is an analog, isomer, metabolite, derivative, pharmaceutically acceptable salt, pharmaceutical product, N-oxide, hydrate or any combination thereof of said compound.

3. The composition according to claim 1, wherein said selective androgen receptor modulator is triphenylethylene, toremifene, or a combination thereof.

4. The composition according to claim 1, wherein said selective estrogen receptor modulator compound is at a concentration of between about 5 to about 80 milligrams.

5. The composition according to claim 1, wherein said 5-alpha reductase inhibitor is dutasteride or finasteride, or a combination thereof.

6. The composition according to claim 1, further comprising a carrier or diluent.

7. The composition according to claim 6, wherin said carrier or diluent is lactose monohydrate, microcrystalline cellulose, or a mixture thereof.

8. The composition according to claim 1, further comprising a lubricant.

9. The composition of claim 8, wherein said lubricant is magnesium stearate.

10. The composition according to claim 1, further comprising a flow-aid.

11. The composition according to claim 10, wherein said flow aid is colloidal silicon dioxide.

12. The composition according to claim 1, further comprising one or more additives selected from a binder, a disintegrant, a buffer, a protease inhibitor, a surfactant, a solubilizing agent, a plasticizer, an emulsifier, a stabilizing agent, a viscosity increasing agent, a sweetner, a film forming agent, or any combination thereof.

13. The composition according to claim 1, wherein said composition is in the form of a pellet, a tablet, a capsule, a solution, a suspension, a dispersion, an emulsion, an elixir, a gel, an ointment, a cream, or a suppository.

14. The composition according to claim 1, wherein said composition is in the form of a capsule.

15. The composition according to claim 1, wherein said composition is in a form suitable for oral, intravenous, intraarterial, intramuscular, subcutaneous, parenteral, transmucosal, transdermal, or topical administration.

16. The composition according to claim 1, wherein said composition is in a form suitable for oral administration.

17. The composition according to claim 1, wherein said composition is a controlled release composition.

18. The composition according to claim 1, wherein said composition is an immediate release composition.

19. The composition according to claim 1, wherein said composition is a liquid dosage form.

20. The composition according to claim 1, wherein said composition is a solid dosage form.

21. A method of hormone therapy comprising the step of administering to said subject the composition of claim 1, in an amount effective to effect a change in an estrogen-dependent condition.

22. A method of hormone replacement therapy comprising the step of administering to said subject the composition of claim 1, in an amount effective to effect a change in an estrogen-dependent condition.

23. A method of suppressing, inhibiting, or reducing the incidence of pre-malignant lesions of prostate cancer in a subject comprising the step of administering to said subject the composition of claim 1, in an amount effective to suppress, inhibit or reduce the incidence of pre-malignant lesions of prostate cancer in the subject.

24. A method of treating a human with pre-malignant lesions of prostate cancer cancer in a subject comprising the step of administering to said subject the composition of claim 1, in an amount effective to treat of pre-malignant lesions of prostate cancer in the subject.

25. The method of claims 23 or 24, wherein said composition comprises about 5 mg of the analog or a metabolite of the compound of formula (I).

26. The method of claims 23 or 24, wherein said composition comprises about 50 mg of the analog or a metabolite of the compound of formula (I).

27. The method of claims 23 or 24, wherein said composition comprises about 100 mg of the analog or a metabolite of the compound of formula (I).

28. The method of claims 23 or 24, wherein said pre-malignant lesion is a precancerous precursor of prostate adenocarcinoma.

29. The method of claim 28, wherein the precancerous precursor of prostate adenocarcinoma is prostate intraepithelial neoplasia (PIN).

30. The method of claim 29, wherein the prostate intraepithelial neoplasia is high grade prostate intraepithelial neoplasia (HGPIN).

31. A method of suppressing, inhibiting, or reducing the incidence of latent prostate cancer in a subject comprising the step of administering to said subject the composition of claim 1, in an amount effective to suppress, inhibit or reduce the incidence of prostate cancer in the subject.

32. A method of treating a subject with latent prostate cancer comprising the step of administering to said subject the composition of claim 1, in an amount effective to treat prostate cancer in the subject.

33. The method of claims 31 or 32, wherein said composition comprises about 20 mg of the analog or a metabolite of the compound of formula (I).

34. The method of claims 31 or 32, wherein said composition comprises about 40 mg of the analog or a metabolite of the compound of formula (I).

35. The method of claims 31 or 32, wherein said composition comprises about 60 mg of the analog or a metabolite of the compound of formula (I).

36. The method of claims 31 or 32, wherein suppressing, inhibiting, reducing the incidence of or treating prostate cancer is via suppressing, inhibiting, reducing the incidence of or treating a precancerous precursor of prostate adenocarcinoma.

37. The method of claim 36, wherein the precancerous precursor of prostate adenocarcinoma is prostate intraepithelial neoplasia (PIN).

38. The method of claim 37, wherein the prostate intraepithelial neoplasia is high grade prostate intraepithelial neoplasia (HGPIN).

39. A method of preventing suppressing, inhibiting, or reducing the incidence of prostate carcinogenesis in a subject comprising the step of administering to said subject the composition of claim 1, in an amount effective to prevent, suppress, inhibit or reduce the incidence of prostate cancer in the subject.

40. The method of claims 39, wherein said composition comprises about 5 mg of the analog or a metabolite of the compound of formula (I).

41. The method of claims 39, wherein said composition comprises about 50 mg of the analog or a metabolite of the compound of formula (I).

42. The method of claims 39, wherein said composition comprises about 100 mg of the analog or a metabolite of the compound of formula (I).

43. The method of claims 39, wherein preventing, suppressing, inhibiting or reducing the incidence of prostate cancer is via preventing, suppressing, inhibiting, reducing the incidence of or treating a precancerous precursor of prostate adenocarcinoma.

44. The method of claim 43, wherein the precancerous precursor of prostate adenocarcinoma is prostate intraepithelial neoplasia (PIN).

45. The method of claim 44, wherein the prostate intraepithelial neoplasia is high-grade prostate intraepithelial neoplasia (HGPIN).

46. A method of treating androgen-deprivation induced osteoporosis in a male subject suffering from prostate cancer, comprising the step of administering to said subject the composition of claim 1, in an amount effective to treat androgen-deprivation induced osteoporosis in said subject.

47. A method of suppressing, inhibiting, reducing the risk of developing or preventing androgen-deprivation induced osteoporosis in a male subject suffering from prostate cancer, comprising the step of administering to said subject the composition of claim 1, in an amount effective to suppress, inhibit, reduce the risk of developing or prevent androgen-deprivation induced osteoporosis in said subject.

48. A method of suppressing, inhibiting, reducing the risk of developing, preventing or treating androgen-deprivation induced loss of bone mineral density (BMD) in a male subject suffering from prostate cancer, comprising the step of administering to said subject the composition of claim 1, in an amount effective to suppress, inhibit, reduce the risk of developing, prevent or treat androgen-deprivation induced bone loss in said subject.

49. A method of suppressing, inhibiting, reducing the risk of developing, preventing or treating androgen-deprivation induced bone fractures in a male subject suffering from prostate cancer, comprising the step of administering to said subject the composition of claim 1, in an amount effective to suppress, inhibit, reduce the risk of developing, prevent or treat androgen-deprivation induced bone fractures in said subject.

50. A method of suppressing, inhibiting, reducing the risk of developing, preventing or treating a subject with hot flashes, comprising the step of administering to said subject the composition of claim 1, in an amount effective to suppress, inhibit, reduce the risk of developing, prevent or treat hot flashes in said subject.

51. The method of claim 50, wherein said subject suffers from prostate cancer and has been exposed to androgen-deprivation therapy.

52. A method of suppressing, inhibiting, reducing the risk of developing, preventing or treating a subject with gynecomastia, comprising the step of administering to said subject the composition of claim 1, in an amount effective to suppress, inhibit, reduce the risk of developing, prevent or treat gynecomastia in said subject.

53. The method of claim 52, wherein said subject suffers from prostate cancer and has been exposed to androgen-deprivation therapy.

54. A method of suppressing, inhibiting, reducing the risk of developing, preventing or treating a subject with endometrial carcinoma, comprising the step of administering to said subject the composition of claim 1, in an amount effective to suppress, inhibit, reduce the risk of developing, prevent or treat endometrial carcinoma in said subject.

55. A method of suppressing, inhibiting, reducing the risk of developing, preventing or treating a subject with polycystic ovarian syndrome, comprising the step of administering to said subject the composition of claim 1, in an amount effective to suppress, inhibit, reduce the risk of developing, prevent or treat polycystic ovarian syndrome in said subject.

56. A method of suppressing, inhibiting, delaying onset or preventing diabetes, breast cancer, endometrial carcinoma or cardiovascular disease in a female subject suffereing from polycystic ovarian syndrome, comprising the step of administering to said subject the composition of claim 1, in an amount effective to suppress, inhibit, delay onset, or prevent diabetes, breast cancer, endometrial carcinoma or cardiovascular disease in the subject.
